(19) United States
(12) Patent Application Publication
(10) Pub. No.: US 2023/0223117 A1
(43) Pub. Date: Jul. 13, 2023

(54) FAMILY INTENT ACQUISITION APPARATUS

(71) Applicant: Canon Medical Systems Corporation, Otawara-shi (JP)

(72) Inventors: Takuya SAKAGUCHI, Utsunomiya (JP); Yasuko FUJISAWA, Nasushiobara (JP)

(73) Assignee: Canon Medical Systems Corporation, Otawara-shi (JP)

(21) Appl. No.: 18/152,798

(22) Filed: Jan. 11, 2023

(30) Foreign Application Priority Data
Jan. 13, 2022 (JP) 2022-003749

(51) Int. Cl. G16H 10/20; G10L 17/22; G10L 25/57

(52) U.S. Cl. CPC G16H 10/20; G10L 17/22; G10L 25/57

(57) ABSTRACT

According to one embodiment, a family intent acquisition apparatus includes processing circuitry. The processing circuitry is configured to acquire an intent of a patient and an intent of a family who cares for or supports the patient. The processing circuitry is configured to assign the intent of the patient with a patient ID, and assign the intent of the family with a family ID. The processing circuitry is configured to transmit intent information including the intent assigned with the patient ID and the intent assigned with the family ID.

### CROSS-REFERENCE TO RELATED APPLICATION

This application is based upon and claims the benefit of priority from the prior Japanese Patent Application No. 2022-003749, filed Jan. 13, 2022, the entire contents of which are incorporated herein by reference.

### FIELD

Embodiments described herein relate generally to a family intent acquisition apparatus.

### BACKGROUND

Recently, it has become mainstream to decide patients' treatment or patients' future life based on their intent. However, if a patient cannot make a decision by him/herself, it is necessary to acquire the intent of the patient's family. For example, if decisions need to be made on indirect matters such as nursing care and costs, it will be difficult for a patient to make decisions by him/herself, which makes it necessary to respect the intent of the patient's family who will take charge of such matters.

On the other hand, medical staff may be unable to properly select treatment because of not knowing whether the information provided by a patient or a family is the patient's intent or the family's intent. Therefore, it is desirable to be able to acquire a patient's intent and a family's intent separately.

### BRIEF DESCRIPTION OF THE DRAWINGS

FIG. 1 is a block diagram showing a configuration of a family intent acquisition system which includes a family intent acquisition apparatus according to a first embodiment.

FIG. 2 is a block diagram showing a configuration of the family intent acquisition apparatus shown in FIG. 1.

FIG. 3 is a block diagram showing a configuration of the medical terminal shown in FIG. 1.

FIG. 4 is a sequence diagram for explaining an operation performed in the first embodiment.

FIG. 5 is a schematic diagram showing an example of an explanatory text used in the operation shown in FIG. 4.

FIG. 6 is a schematic diagram showing an example of an explanatory text with special notes used in the operation shown in FIG. 4.

FIG. 7 is a block diagram showing a configuration of a family intent acquisition system according to a first modification of the first embodiment.

FIG. 8 is a block diagram showing a configuration of the AI processing apparatus shown in FIG. 7.

FIG. 9 is a sequence diagram for explaining an operation of the family intent acquisition system shown in FIG. 7.

FIG. 10 is a block diagram showing a configuration of a family intent acquisition system according to a third modification of the first embodiment.

FIG. 11 is a sequence diagram for explaining an operation of the family intent acquisition system shown in FIG. 10.

FIG. 12 is a block diagram showing a configuration of a family intent acquisition system according to a fourth modification of the first embodiment.

FIG. 13 is a sequence diagram for explaining an operation of the family intent acquisition system shown in FIG. 12.

FIG. 14 is a flowchart for explaining an operation of a family intent acquisition system according to a second embodiment.

FIG. 15 is a schematic diagram showing an example of a display screen for explaining the operation shown in FIG. 14.

FIG. 16 is a schematic diagram showing a modification of the display screen for explaining the operation shown in FIG. 14.

FIG. 17 is a schematic diagram showing another modification of the display screen for explaining the operation shown in FIG. 14.

FIG. 18 is a schematic diagram showing still another modification of the display screen for explaining the operation shown in FIG. 14.

FIG. 19 is a flowchart for explaining an operation of a family intent acquisition system according to a third embodiment.

FIG. 20 is a schematic diagram for explaining the operation performed in the third embodiment.

FIG. 21 is a schematic diagram for explaining a modification of a voice-input device used in the operation shown in FIG. 20.

FIG. 22 is a flowchart for explaining a first modification of the operation shown in FIG. 20.

FIG. 23 is a schematic diagram for explaining the operation shown in FIG. 22.

FIG. 24 is a schematic diagram for explaining a modification of voiceprint recognition performed in the operation shown in FIG. 22.

FIG. 25 is a flowchart for explaining a second modification of the operation shown in FIG. 20.

FIG. 26 is a schematic diagram for explaining the operation shown in FIG. 25.

FIG. 27 is a flowchart for explaining a third modification of the operation shown in FIG. 20.

FIG. 28 is a schematic diagram for explaining the operation shown in FIG. 27.

FIG. 29 is a schematic diagram showing an example of a display screen for explaining the operation shown in FIG. 27.

FIG. 30 is a block diagram showing a configuration of a family intent acquisition system according to a fourth embodiment.

FIG. 31 is a sequence diagram for explaining an operation of the family intent acquisition system shown in FIG. 30.

FIG. 32 is a schematic diagram for explaining the operation shown in FIG. 31.

FIG. 33 is a block diagram showing a configuration of a family intent acquisition apparatus according to a fifth embodiment.

FIG. 34 is a sequence diagram for explaining an operation performed in the fifth embodiment.

FIG. 35 is a schematic diagram showing an example of a display screen for explaining the operation shown in FIG. 34.

FIG. 36 is a schematic diagram showing another example of a display screen for explaining the operation shown in FIG. 34.

FIG. 37 is a schematic diagram showing still another example of a display screen for explaining the operation shown in FIG. 34.

### DETAILED DESCRIPTION

In general, according to one embodiment, a family intent acquisition apparatus includes processing circuitry. The processing circuitry is configured to acquire an intent of a patient and an intent of a family who cares for or supports the patient. The processing circuitry is configured to assign the intent of the patient with a patient ID, and assign the intent of the family with a family ID. The processing circuitry is configured to transmit intent information including the intent assigned with the patient ID and the intent assigned with the family ID.

Hereinafter, the embodiments will be described with reference to the drawings. The term “family” as used herein refers to a person who cares for or supports a patient and is not limited to a person living with a patient or a person related to a patient by blood. Namely, the “family” includes, for example, a guardian for a patient and people who take care of a patient.

### First Embodiment

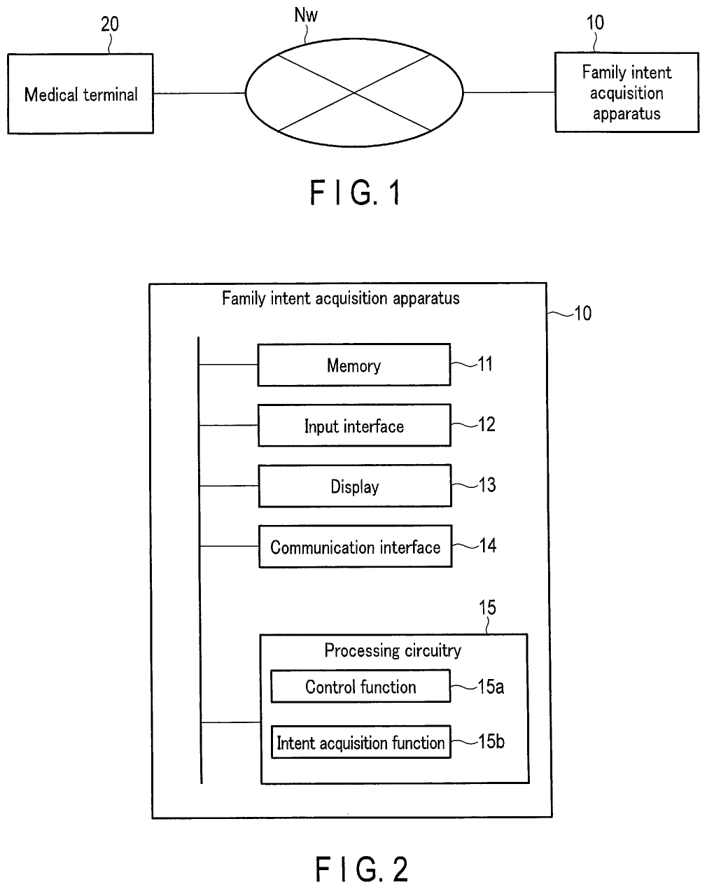

FIG. 1 is a block diagram showing a configuration of a family intent acquisition system which includes a family intent acquisition apparatus according to a first embodiment. A family intent acquisition apparatus 10 is a terminal apparatus which is used by a patient or a patient's family and is capable of communicating with a medical terminal 20 via a communication network Nw such as the Internet. For example, a mobile terminal, such as a smartphone, a tablet terminal, or a laptop computer, or an installed terminal, such as a desktop computer, may be used as the family intent acquisition apparatus 10.

The family intent acquisition apparatus 10 includes a memory 11, an input interface 12, a display 13, a communication interface 14, and processing circuitry 15, as shown in FIG. 2. The memory 11, the input interface 12, the display 13, the communication interface 14, and the processing circuitry 15 are, for example, communicatively connected to one another via a bus.

The memory 11 is constituted by a memory for storing electronic information, as exemplified by a read only memory (ROM), a random access memory (RAM), a hardware disk drive (HDD), an image memory, etc., and peripheral circuitry that accompanies the memory, as exemplified by a memory controller, a memory interface, etc. The memory 11 stores, for example, various programs such as an intent acquisition program of the family intent acquisition apparatus 10, various tables, and various pieces of data such as data being processed and data after being processed.

The input interface 12 is implemented by components for inputting various instructions, commands, information pieces, selections, and settings from an operator (user) to the main unit of the family intent acquisition apparatus, and such components include a trackball, switch buttons, a mouse, a keyboard, or a touchpad (or a trackpad) which enables performance of input operations through contact with the operation screen, a main unit microphone which enables performance of voice input operations, and a touch panel display (or a touch screen) which integrates a display screen and a touchpad. The input interface 12 is connected to the processing circuitry 15. The input interface 12 converts input operations received from a user into electric signals and outputs the electric signals to the processing circuitry 15. In this case, the input interface 12 may cause the display 13 to display a user interface (graphical user interface (GUI)) through which a user inputs various instructions using physical operation components such as a mouse and a keyboard. In the disclosure herein, the input interface 12 is not limited to physical operation components. Examples of the input interface 12 also include processing circuitry for electric signals, which receives electric signals corresponding to input operations from an external input device (such as an external microphone, an external wearable terminal, etc.) separate from its own apparatus, and outputs the electric signals to the processing circuitry 15. In the description below, “an operation of the input interface 12 performed by a user” is also referred to as “a user operation”.

The display 13 is constituted by a display main part for displaying various screens, internal circuitry for supplying signals for display to the display main part, and peripheral circuitry including connectors, cables, etc., for connecting the display with the internal circuitry. The display 13 is capable of displaying any data such as a patient's entry field, a family's entry field, an entered content, an icon, a GUI, etc., as appropriate. The display 13 is an example of a display unit.

The communication interface 14 is circuitry for connecting the family intent acquisition apparatus 10 to a network for communication with other apparatuses. For example, a network interface card (NIC) may be used as the communication interface 14. A description regarding the communication interface 14 being involved in the communication between the family intent acquisition apparatus 10 and other apparatuses will be omitted below.

The processing circuitry 15 reads the programs stored in the memory 11 based on the instructions input by the user through the input interface 12 and controls the family intent acquisition apparatus 10 based on the programs. For example, the processing circuitry 15 is a processor to implement various functions of the family intent acquisition apparatus 10 according to the various programs read from the memory 11. The various functions include, for example, a control function 15a and an intent acquisition function 15b. The various functions may be implemented by multiple processors separately, as necessary. Alternatively, all or some of the functions may be implemented by other apparatuses, as necessary. For example, a voiceprint recognition function (described later) included in the intent acquisition function 15b, and the like, among the various functions, may be implemented by other apparatuses (not shown).

The control function 15a, which is a usual computer function, is mainly a function other than the intent acquisition function 15b, but includes a peripheral function of the intent acquisition function 15b. Examples of the peripheral function of the intent acquisition function 15b include a transmission function of transmitting intent information including an intent given a patient ID by the intent acquisition function 15b and an intent given a family ID by the intent acquisition function 15b. Examples of the peripheral function of the intent acquisition function 15b also include a display control function of causing the display 13 to display a patient entry field for entering a patient's intent and a family entry field for entering a family's intent. The control function 15a is an example of a transmitter and a display controller.

The intent acquisition function 15b acquires the intent of a patient and the intent of a family who cares for or supports the patient. For example, the intent acquisition function 15b acquires a content entered into the patient entry field as a patient's intent, and acquires a content entered into the family entry field as a family's intent. The intent acquisition function 15b also assigns a patient ID to the patient's intent and assigns a family ID to the family's intent. The intent acquisition function 15b is an example of an acquisition unit and an assignment unit.

On the other hand, the medical terminal 20 is a terminal device which is used by a doctor or medical staff taking charge of patient's treatment and is capable of communicating with the family intent acquisition apparatus 10 via the communication network Nw such as the Internet. For example, a mobile terminal, such as a smartphone, a tablet terminal, or a laptop computer, or an installed terminal, such as a desktop computer, may be used as the medical terminal 20.

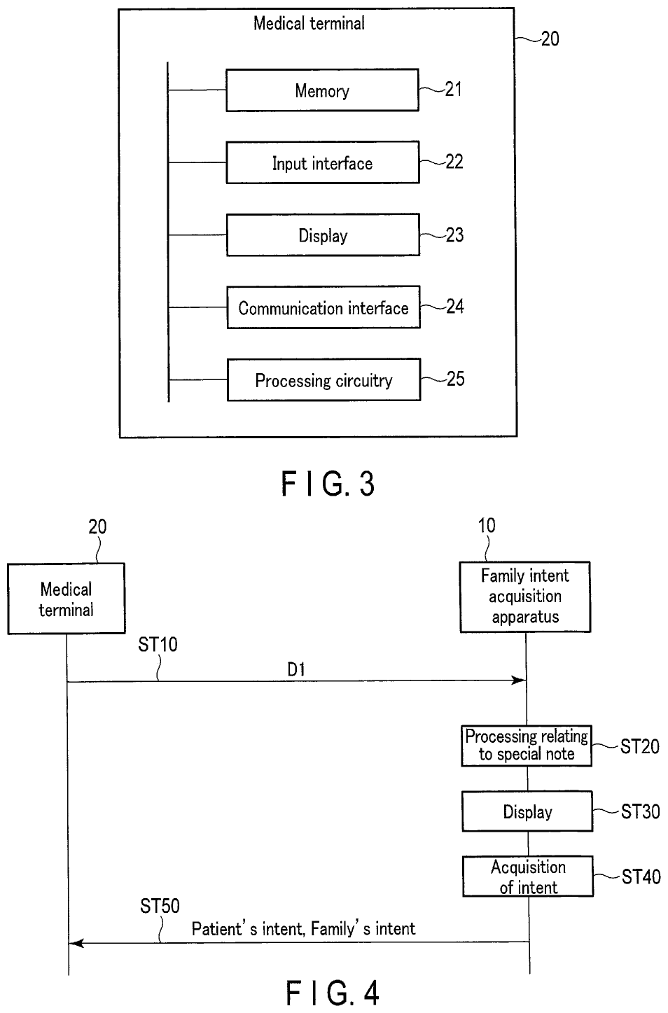

The medical terminal 20 includes a memory 21, an input interface 22, a display 23, a communication interface 24, and processing circuitry 25, as shown in FIG. 3. The memory 21, the input interface 22, the display 23, the communication interface 24, and the processing circuitry 25 are, for example, communicatively connected to one another via a bus.

The memory 21 is constituted by a memory for storing electronic information, as exemplified by a ROM, a RAM, an HDD, an image memory, etc., and peripheral circuitry that accompanies the memory, as exemplified by a memory controller, a memory interface, etc. The memory 21 stores, for example, various programs of the medical terminal 20, various tables, and various pieces of data such as data being processed and data after being processed.

The input interface 22 is implemented by components for inputting various instructions, commands, information pieces, selections, and settings from an operator (user) to the main unit of the family intent acquisition apparatus, and such components include a trackball, switch buttons, a mouse, a keyboard, a touchpad (or a trackpad) which enables performance of input operations through contact with the operation screen, a main unit microphone which enables performance of voice input operations, and a touch panel display (or a touch screen) which integrates a display screen and a touchpad. The input interface 22 is connected to the processing circuitry 25. The input interface 22 converts input operations received from a user into electric signals and outputs the electric signals to the processing circuitry 25. In this case, the input interface 22 may cause the display 23 to display a user interface (graphical user interface (GUI)) through which a user inputs various instructions using physical operation components such as a mouse and a keyboard. In the disclosure herein, the input interface 22 is not limited to physical operation components. Examples of the input interface 22 also include processing circuitry for electric signals, which receives electric signals corresponding to input operations from an external input device (such as an external microphone, an external wearable terminal, etc.) separate from its own apparatus, and outputs the electric signals to the processing circuitry 25. In the description below, “an operation of the input interface 22 performed by a user” is also referred to as “a user operation”.

The display 23 is constituted by a display main part for displaying various screens, internal circuitry for supplying signals for display to the display main part, and peripheral circuitry including connectors, cables, etc., for connecting the display with the internal circuitry. The display 23 is capable of displaying any data such as a patient's entry field, a family's entry field, an entered content, an icon, a GUI, etc., as appropriate. The display 13 is an example of a display unit.

The communication interface 24 is circuitry for connecting the medical terminal 20 to the communication network Nw for communication with other apparatuses. For example, a network interface card (NIC) may be used as the communication interface 24. A description regarding the communication interface 24 being involved in the communication between the medical terminal 20 and other apparatuses will be omitted below.

The processing circuitry 25 reads the programs stored in the memory 21 based on the instructions input by the user through the input interface 22 and controls the medical terminal 20 based on the programs. For example, the processing circuitry 25 is a processor to implement various functions of the medical terminal 20 according to the various programs read from the memory 21. The various functions include, for example, ordinary computer functions, a function of performing processing relating to a special note on an explanatory text provided to a patient and a family, and the like. The various functions may be implemented by multiple processors separately, as necessary. Alternatively, all or some of the functions may be implemented by other apparatuses, as necessary. For example, the function of performing processing relating to a special note, and the like, among the various functions, may be implemented by other apparatuses (not shown).

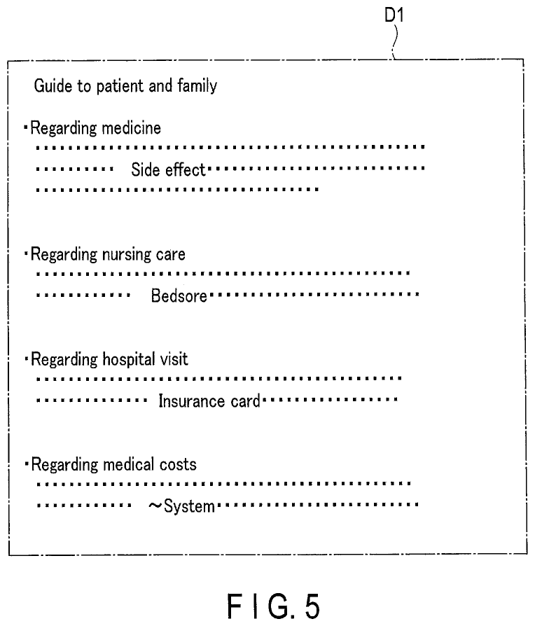

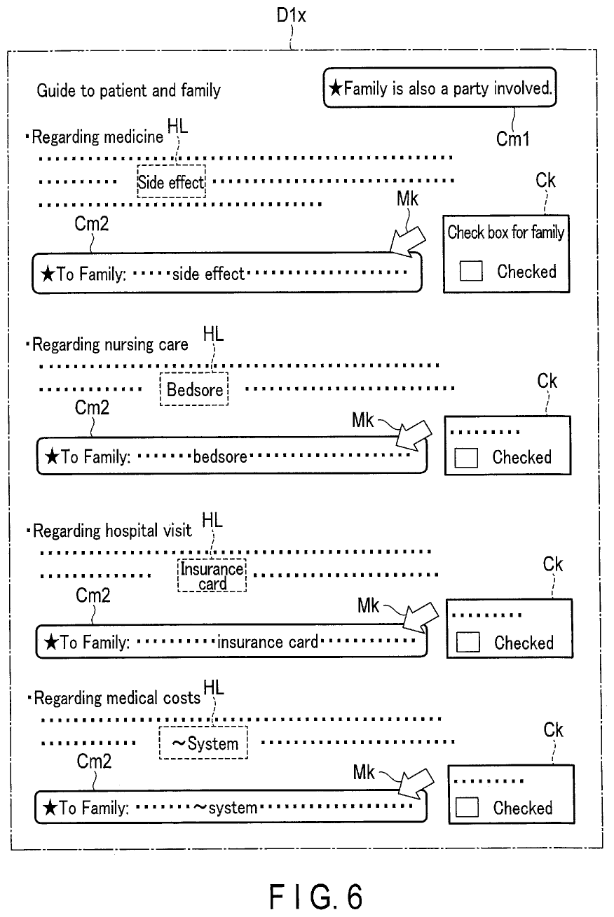

Next, an operation of the family intent acquisition system provided with the family intent acquisition apparatus configured as above will be described with reference to the sequence diagram shown in FIG. 4 and the schematic diagrams shown in FIGS. 5 and 6.

It is assumed that the medical terminal 20 is making an explanatory text D1 for a patient and a family according to an operation performed by medical staff, as shown in FIG. 5, and storing the explanatory text D1 in the memory 21 now. The explanatory text D1 includes general explanations regarding medicine, nursing care, hospital visits, medical care, and the like for the patient and the family, but does not include a description stating that the family is a party concerned. In this state, step ST10 is started.

In step ST10, the processing circuitry 25 of the medical terminal 20 transmits the explanatory text D1 to the family intent acquisition apparatus 10 according to an operation performed by medical staff. The family intent acquisition apparatus 10 receives the explanatory text D1 and stores it in the memory 11.

In step ST20 after step ST10, the processing circuitry 15 of the family intent acquisition apparatus 10 reads the explanatory text D1 stored in the memory 11 and performs the processing relating to a special note on the explanatory text D1. The processing relating to a special note is processing of adding a special note for all or some of the items in the explanatory text D1 with which the family is concerned and notifying the family. Specifically, a fixed sentence Cm1, for example, may be added to the explanatory text D1. For example, processing of highlighting an item of a special note or a description or processing of adding a fixed sentence Cm2 regarding an item of a special note may also be suitably adopted as the processing relating to a special note, as shown in FIG. 6. Examples of the processing of highlighting an item of a special note or a description include use of a character decoration such as a highlight HL, addition of a mark Mk to the item of a special note or the fixed sentence Cm2, and the like. As the processing relating to a special note, for example, a check box Ck for confirming that the fixed sentence Cm2, etc., have been seen may be added. Alternatively, a message that prompts attachment of a predetermined image such as a mark indicating that the fixed sentence Cm has been seen may be added in place of the check box Ck.

Specifically, the processing circuitry 15, for example, first extracts an item of a special note from the explanatory text D1. As the item of a special note, for example, desired items such as “Side effects” in the explanation of medicine, “Bedsores” in the explanation of nursing care, “Insurance card” in the explanation of hospital visits, and “˜System” in the explanation of medical costs may be suitably used.

Thereafter, the processing circuitry 15 adds the highlight HL, fixed sentence Cm2, mark Mk, and check box Ck based on the extracted item. The processing circuitry 15 also adds the fixed sentence Cm1 to the entire explanatory text D1. In this manner, the processing circuitry 15 performs the processing relating to a special note on the explanatory text D1 to produce an explanatory text D1x.

In step ST30 after step ST20, the processing circuitry 15 causes the display 13 to display the produced explanatory text D1x. The processing circuitry 15 causes the display 13 to display an intent entry screen (not shown) for the explanatory text D1x.

In step ST40 after step ST30, the processing circuitry 15 acquires a patient's intent according to an operation performed on the entry screen by the patient. The processing circuitry 15 updates the check box Ck of the explanatory text D1x according to an operation performed on the explanatory text D1x by the family, and acquires the family's intent according to an operation performed on the entry screen by the family. Thereafter, the processing circuitry 15 assigns a patient ID to the acquired patient's intent and assigns a family ID to the acquired family's intent.

In step ST50 after step ST40, the processing circuitry 15 transmits, to the medical terminal 20, intent information including the explanatory text D1x with the check box Ck updated, the intent given the patient ID, and the intent given the family ID. However, the explanatory text D1x with the check box Ck updated may be omitted because it may be obvious from the family's intent that the family saw the fixed sentence Cm2.

After step ST50, the medical terminal 20 causes the display 23 to display the patient's intent and the family's intent in such a manner as to distinguish them based on the information on the intent received. Thus, medical staff can understand the patient's intent and the family's intent separately.

As described above, according to the first embodiment, the intent of a patient and the intent of the family who cares for or supports the patient are acquired. In addition, the patient's intent is assigned with a patient ID, and the family's intent is assigned with a family ID. Also, intent information including the intent assigned with a patient ID and the intent assigned with a family ID is transmitted. In this manner, the patient's intent and the family's intent can be acquired separately.

(Modifications of First Embodiment)

The first embodiment may be implemented in the manner shown in a first modification to a fourth modification described below.

(First Modification of First Embodiment)

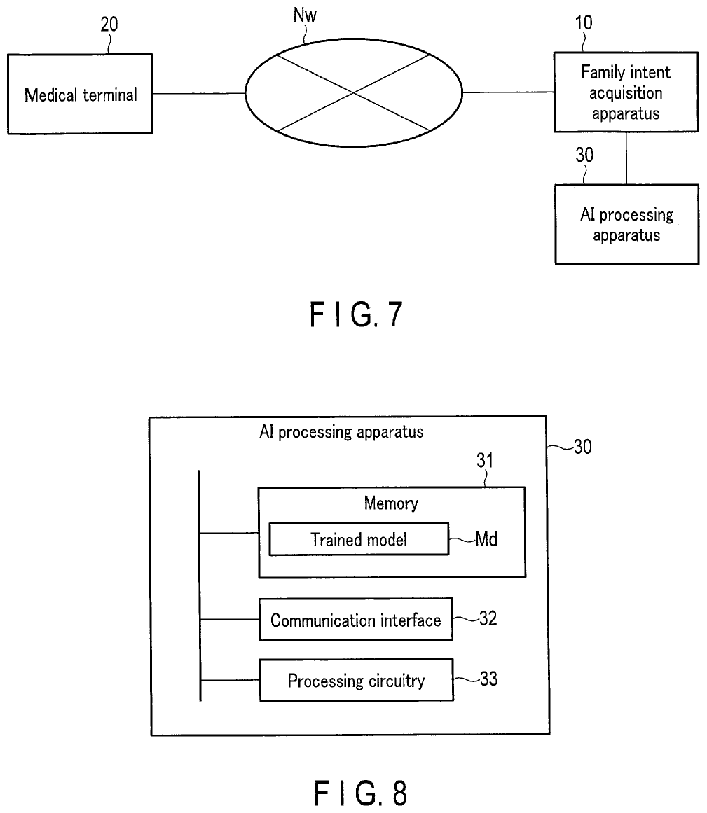

FIG. 7 is a block diagram showing a configuration of a family intent acquisition system according to a first modification of the first embodiment. The same reference symbols are used for substantially the same components as those shown in FIG. 1, and a detailed description of those components will be omitted. The description below focuses mainly on the aspects differing from FIG. 1. Also, for the respective modifications and respective embodiments shown below, redundant explanations will likewise be omitted.

In the first modification, when the processing relating to a special note is performed on the explanatory text D1 to produce the explanatory text D1x, the processing of extracting an item of a special note from the explanatory text D1 is performed with an AI processing apparatus 30 capable of wireless communication taking charge of part of the processing. Specifically, the family intent acquisition apparatus 10 is equipped with an AI processing apparatus 30 capable of wireless communication, as shown in FIG. 7.

The AI processing apparatus 30 includes a memory 31, a communication interface 32, and processing circuitry 33, as shown in FIG. 8.

The memory 31 is constituted by a memory for storing electronic information, as exemplified by a ROM, a RAM, an HDD, an image memory, etc., and peripheral circuitry that accompanies the memory, as exemplified by a memory controller, a memory interface, etc. The memory 31 stores, for example, various programs of the AI processing apparatus 30, a trained model, various tables, and various pieces of data such as data being processed and data after being processed. The trained model is a neural network which is machine-trained so as to generate an item with which a family is concerned from the explanatory text D1 based on the explanatory text D1.

The communication interface 32 is circuitry for connecting the AI processing apparatus 30 to the communication network Nw for communication with other apparatuses. For example, a network interface card (NIC) may be used as the communication interface 32. A description regarding the communication interface 32 being involved in the communication between the AI processing apparatus 30 and other apparatuses will be omitted below.

The processing circuitry 33 is a processor which reads the programs stored in the memory 31 and controls the AI processing apparatus 30 based on the programs. Upon receiving the explanatory text D1 from the communication network Nw, for example, the processing circuitry 33 inputs the explanatory text D1 into the trained model that generates an item of a special note with which a family is concerned from the explanatory text D1 based on the explanatory text D1, thereby generating an item of a special note with which a family is concerned. Also, the processing circuitry 33 returns the generated result to the transmitter of the explanatory text D1.

Accordingly, the processing circuitry 15 of the family intent acquisition apparatus 10 transmits the explanatory text D1 received from the medical terminal 20 to the AI processing apparatus 30, and adds the highlight HL, fixed sentence Cm2, mark Mk, and check box Ck based on the item returned from the AI processing apparatus 30. The processing circuitry 15 adds the fixed sentence Cm1 to the entire explanatory text D1. In this manner, the processing circuitry 15 performs the processing relating to a special note on the explanatory text D1 to produce the explanatory text D1x.

The remaining aspects are the same as the first embodiment.

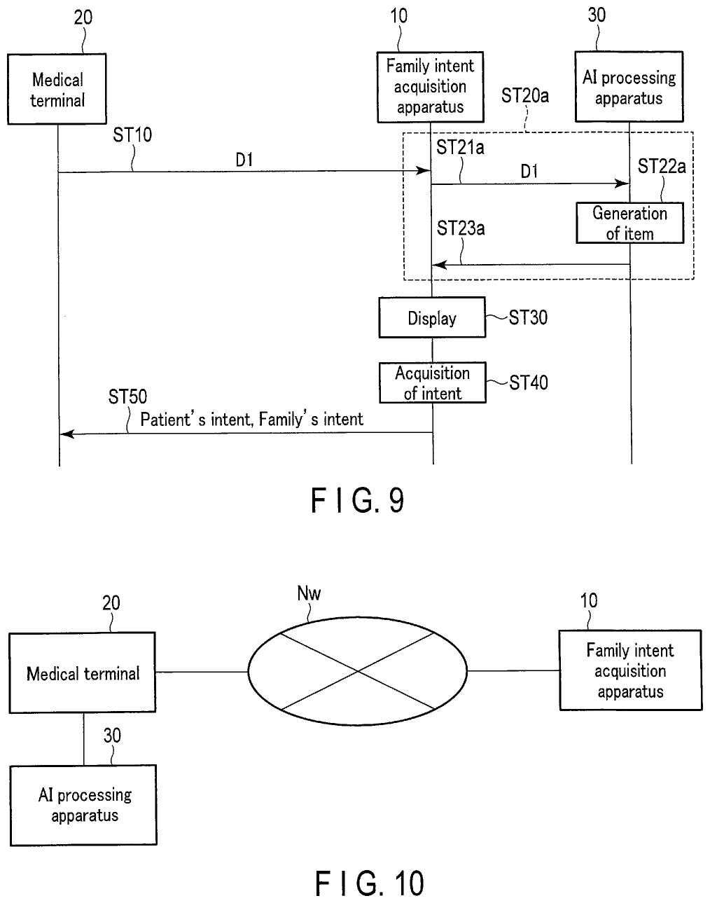

The first modification described above differs from the first embodiment in that an operation of step ST20a consisting of steps ST21a to ST23a is performed in place of step ST20, as shown in FIG. 9. For example, step ST10 is performed in the same manner as described above, and the processing circuitry 15 of the family intent acquisition apparatus 10 receives the explanatory text D1 and stores it in the memory 11.

The processing circuitry 15 transmits the explanatory text D1 to the AI processing apparatus 30 (step ST21a).

Upon receiving the explanatory text D1, the AI processing apparatus 30 generates an item of a special note with which a family is concerned from the explanatory text D1 based on the explanatory text D1 (step ST22a), and returns the generated result to the family intent acquisition apparatus 10 (step ST23a).

The processing circuitry 15 of the family intent acquisition apparatus 10 adds the highlight HL, fixed sentence Cm2, mark Mk, and check box Ck based on the item returned from the AI processing apparatus 30. The processing circuitry 15 adds the fixed sentence Cm1 to the entire explanatory text D1. In this manner, the processing circuitry 15 performs the processing relating to a special note on the explanatory text D1 to produce the explanatory text D1x.

Thereafter, the processing of step ST30 and the processing of the subsequent steps are performed in the same manner as described above.

According to the first modification described above, when the processing relating to a special note is performed on the explanatory text D1 to produce the explanatory text D1x, the processing of extracting an item of a special note from the explanatory text D1 can be performed with the AI processing apparatus 30 capable of wireless communication taking charge of part of the processing.

(Second Modification of First Embodiment)

In the second modification, the process for performing the processing relating to a special note on the explanatory text D1 to produce the explanatory text D1x is performed with the medical terminal 20 taking charge of part of the process. Specifically, the medical terminal 20 operated by a doctor or medical staff is capable of communicating with the family intent acquisition apparatus 10 via the communication network Nw, as shown in FIG. 1.

Accordingly, the processing circuitry 25 of the medical terminal 20, for example, produces the explanatory text D1 according to an operation performed by medical staff and stores the explanatory text D1 in the memory 21. The processing circuitry 25 also reads the explanatory text D1 stored in the memory 21 and performs the processing relating to a special note on the explanatory text D1. The processing relating to a special note is processing of adding a special note for all or some of the items in the explanatory text D1 with which the family is concerned and notifying the family. Specifically, the processing circuitry 25 extracts an item of a special note from the explanatory text D1 and adds the highlight HL, fixed sentence Cm2, mark Mk, and check box Ck based on the extracted item in the same manner as described above. The processing circuitry 25 adds the fixed sentence Cm1 to the entire explanatory text D1. In this manner, the processing circuitry 25 performs the processing relating to a special note on the explanatory text D1 to produce the explanatory text D1x. The processing circuitry 25 also transmits the produced explanatory text D1x to the family intent acquisition apparatus 10 according to an operation performed by medical staff.

The remaining aspects are the same as the first embodiment.

According to the second modification described above, the process for performing the processing relating to a special note on the explanatory text D1 to produce the explanatory text D1x can be performed with the medical terminal 20 taking charge of part of the process.

(Third Modification of First Embodiment)

The third modification is a combination of the first modification and the second modification. That is, in the third modification, when the processing relating to a special note is performed on the explanatory text D1 on the medical terminal 20 side to produce the explanatory text D1x, the processing of extracting an item of a special note from the explanatory text D1 is performed with the AI processing apparatus 30 capable of wireless communication taking charge of part of the processing, as shown in FIG. 10. The configuration of the AI processing apparatus 30 is the same as that of the first modification.

Accordingly, the processing circuitry 25 of the medical terminal 20, for example, produces the explanatory text D1 according to an operation performed by medical staff and stores the explanatory text D1 in the memory 21. Also, the processing circuitry 25 transmits the explanatory text D1 stored in the memory 21 to the AI processing apparatus 30, and adds the highlight HL, fixed sentence Cm2, mark Mk, and check box Ck based on the item returned from the AI processing apparatus 30. The processing circuitry 25 adds the fixed sentence Cm1 to the entire explanatory text D1. In this manner, the processing circuitry 25 performs the processing relating to a special note on the explanatory text D1 to produce the explanatory text D1x. The processing circuitry 25 also transmits the produced explanatory text D1x to the family intent acquisition apparatus 10 according to an operation performed by medical staff.

The remaining aspects are the same as the first embodiment.

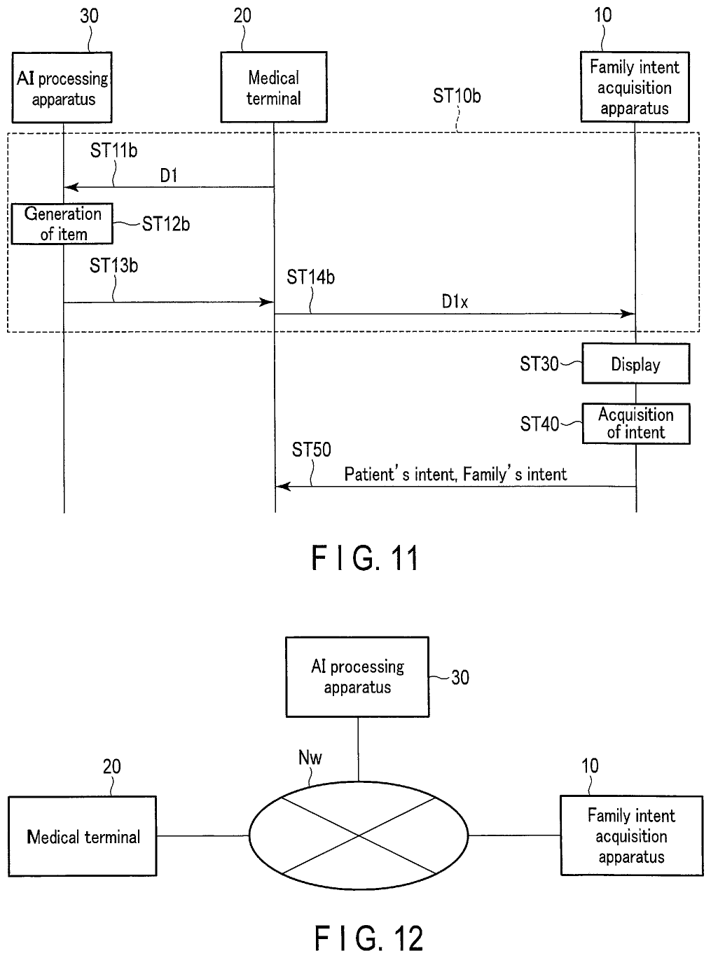

The third modification described above differs from the first embodiment in that an operation of step ST10b consisting of steps ST11b to ST14b is performed in place of step ST10, as shown in FIG. 11.

The processing circuitry 25 of the medical terminal 20, for example, produces the explanatory text D1 according to an operation performed by medical staff and stores the explanatory text D1 in the memory 21. The processing circuitry 25 also transmits the explanatory text D1 stored in the memory 21 to the AI processing apparatus 30 (step ST 11b).

In the same manner as described above, the AI processing apparatus 30 generates an item of a special note with which a family is concerned from the explanatory text D1 based on the explanatory text D1 (step ST12b), and returns the generated result to the medical terminal 20 (step ST13b).

The processing circuitry 25 of the medical terminal 20 performs the processing relating to a special note on the explanatory text D1 based on the item returned from the AI processing apparatus 30, and produces the explanatory text D1x. The processing circuitry 25 also transmits the produced explanatory text D1x to the family intent acquisition apparatus 10 according to an operation performed by medical staff (step ST14b). Thereby, step ST10b consisting of steps ST11b to ST14b is completed.

Thereafter, the processing of step ST30 and the processing of the subsequent steps are performed in the same manner as described above.

According to the third modification described above, when the processing relating to a special note is performed on the explanatory text D1 on the medical terminal 20 side to produce the explanatory text D1x, the processing of extracting an item of a special note from the explanatory text D1 can be performed with the AI processing apparatus 30 capable of wireless communication taking charge of part of the processing.

(Fourth Modification of First Embodiment)

In the fourth modification, the AI processing apparatus 30 is arranged on the communication network Nw between the medical terminal 20 and the family intent acquisition apparatus 10, as shown in FIG. 12. Specifically, in the fourth modification, the AI processing apparatus 30 on the communication network Nw performs the processing relating to a special note on the explanatory text D1 transmitted from the medical terminal 20, produces the explanatory text D1x, and transmits the explanatory text D1x to the family intent acquisition apparatus 10. The AI processing apparatus 30 is operated by a communications company that manages the communication network Nw and functions to receive communications from the medical terminal 20 on the communication network Nw to the family intent acquisition apparatus 10.

Accordingly, the processing circuitry 33 of the AI processing apparatus 30 performs addition of the highlight HL, fixed sentence Cm2, mark Mk, and check box Ck to the explanatory text D1 based on the generated item, in addition to the function of generating an item of a special note based on the explanatory text D1 described above. The processing circuitry 33 adds the fixed sentence Cm1 to the entire explanatory text D1. In this manner, the processing circuitry 33 performs the processing relating to a special note on the explanatory text D1 to produce the explanatory text D1x. The processing circuitry 33 transmits the produced explanatory text D1x to the family intent acquisition apparatus 10.

The remaining aspects are the same as the first embodiment.

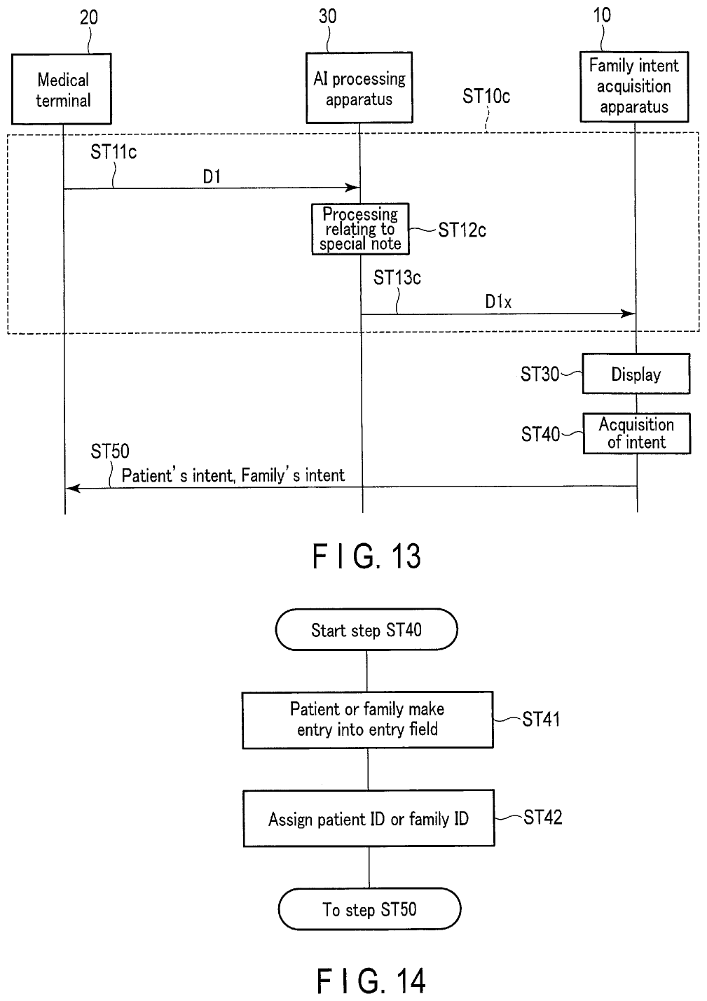

The fourth modification described above differs from the first embodiment in that an operation of step ST10c consisting of steps ST11c to ST13c is performed in place of step ST10, as shown in FIG. 13.

The processing circuitry 25 of the medical terminal 20, for example, produces the explanatory text D1 according to an operation performed by the medical staff and stores the explanatory text D1 in the memory 21. The processing circuitry 25 also transmits the explanatory text D1 stored in the memory 21 to the family intent acquisition apparatus 10 (step ST11c).

The AI processing apparatus 30 receives the explanatory text D1 transmitted to the family intent acquisition apparatus 10, and then generates an item of a special note with which a family is concerned from the explanatory text D1 based the explanatory text D1, followed by performance of the processing relating to a special note on the explanatory text D1 based on the generated item to produce the explanatory text D1x, in the same manner as described above (step ST12c). Thereafter, the AI processing apparatus 30 transmits the produced explanatory text D1x to the family intent acquisition apparatus 10 (step ST13c). Thereby, step ST10c consisting of steps ST11c to ST13c is completed.

Thereafter, the processing of step ST30 and the processing of the subsequent steps are performed in the same manner as described above.

According to the fourth modification described above, the AI processing apparatus 30 on the communication network can perform the processing relating to a special note on the explanatory text D1 transmitted from the medical terminal 20, produce the explanatory text D1x, and transmit the explanatory text D1x to the family intent acquisition apparatus 10.

### Second Embodiment

Next, a family intent acquisition system which includes a family intent acquisition apparatus according to a second embodiment will be described.

The second embodiment is not limited to the instance of presenting the explanatory text D1x described above but includes an aspect of acquiring a patient's intent and a family's intent separately.

Accordingly, the control function 15a of the processing circuitry 15 of the family intent acquisition apparatus 10 causes the display 13 to display a patient entry field for entering a patient's intent and a family entry field for entering a family's intent. The control function 15a is an example of a display controller.

The intent acquisition function 15b of the processing circuitry 15 acquires a content entered into the patient entry field as a patient's intent, and acquires a content entered into the family entry field as a family's intent.

The remaining aspects are the same as the first embodiment. The second embodiment may be applied to each modification of the first embodiment.

According to the above configuration, it is assumed that steps ST10 to ST30 are performed in the same manner as described above and that an intent entry screen for the explanatory text D1x is displayed on the display 13. Specifically, in step ST30, the processing circuitry 15 causes the display 13 to display a patient entry field for entering a patient's intent and a family entry field for entering a family's intent.

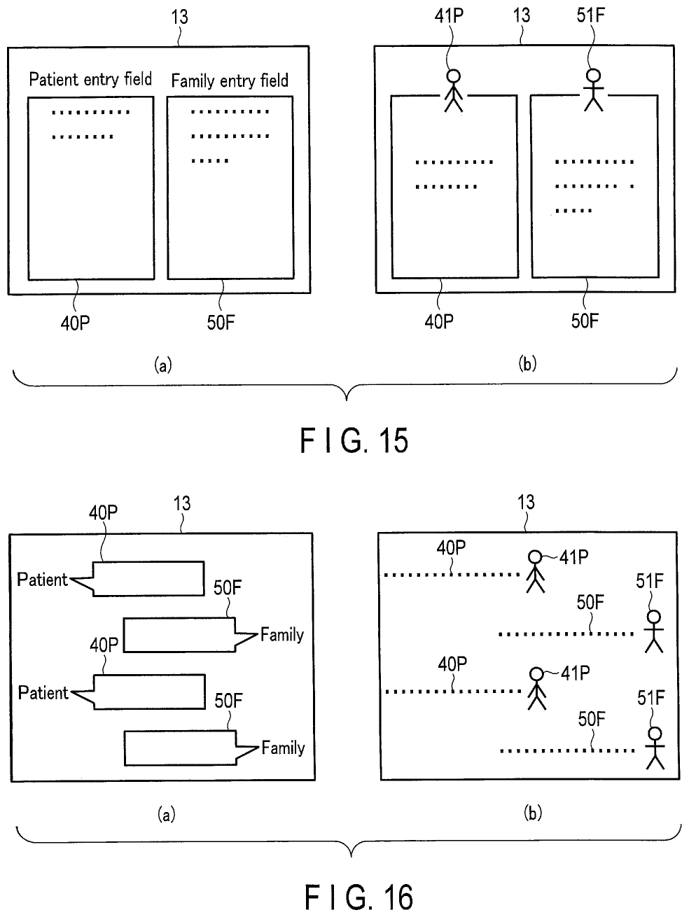

After step ST30, step ST40 consisting of steps ST41 to ST42 is performed, as shown in FIG. 14. It is assumed that according to an operation performed by a patient or a family, the processing circuitry 15 allows the patient or the family to make an entry into the entry field (step ST41). As the entry field, a patient entry field 40P and a family entry field 50F are displayed on the display 13 in such a manner as to be distinguished from each other by a character string, as shown in FIG. 15(a), for example. Alternatively, the patient entry field 40P and the family entry field 50F may be displayed on the display 13 in such a manner as to be distinguished from each other by a patient icon 41P and a family icon 51F, as shown in FIG. 15(b), for example. The patient icon 41P and the family icon 51F have the same color and different shapes. In either case, the processing circuitry 15 acquires a content entered into the patient entry field 40P as a patient's intent, and acquires a content entered into the family entry field 50F as a family's intent.

In step ST42 after step ST41, the processing circuitry 15 assigns a patient ID to the acquired patient's intent and assigns a family ID to the acquired family's intent. Thereby, step ST40 consisting of steps ST41 to ST42 is completed.

Thereafter, the processing of step ST50 and the processing of the subsequent steps are performed in the manner as described.

As described above, according to the second embodiment, a patient entry field for entering a patient's intent and a family entry field for entering a family's intent are displayed on the display 13. The content entered into the patient entry field is acquired as a patient's intent, and the content entered into the family entry field is acquired as a family's intent. Therefore, in addition to the effects achieved by the first embodiment, it is possible to acquire a patient's intent and a family's intent separately based on the content entered into the patient entry field and the content entered into the family entry field.

(Modification of Second Embodiment)

In the second embodiment, a single patient entry field 40P and a single family entry field 50F are displayed; however, the embodiment is not limited thereto. For example, a plurality of patient entry fields 40P and a plurality of family entry fields 50F may be displayed, as shown in FIG. 16(a). When a plurality of patient entry fields 40P and a plurality of family entry fields 50F are displayed, the patient icon 41P may be displayed near each of the patient entry fields 40P closer to the left side of the screen, and the family icon 51F may be displayed near each of the family entry fields 50F closer to the right side of the screen, as shown in FIG. 16(b), for example. Unlike the second embodiment, the patient icon 41P and the family icon 51F may have the same shape and different colors.

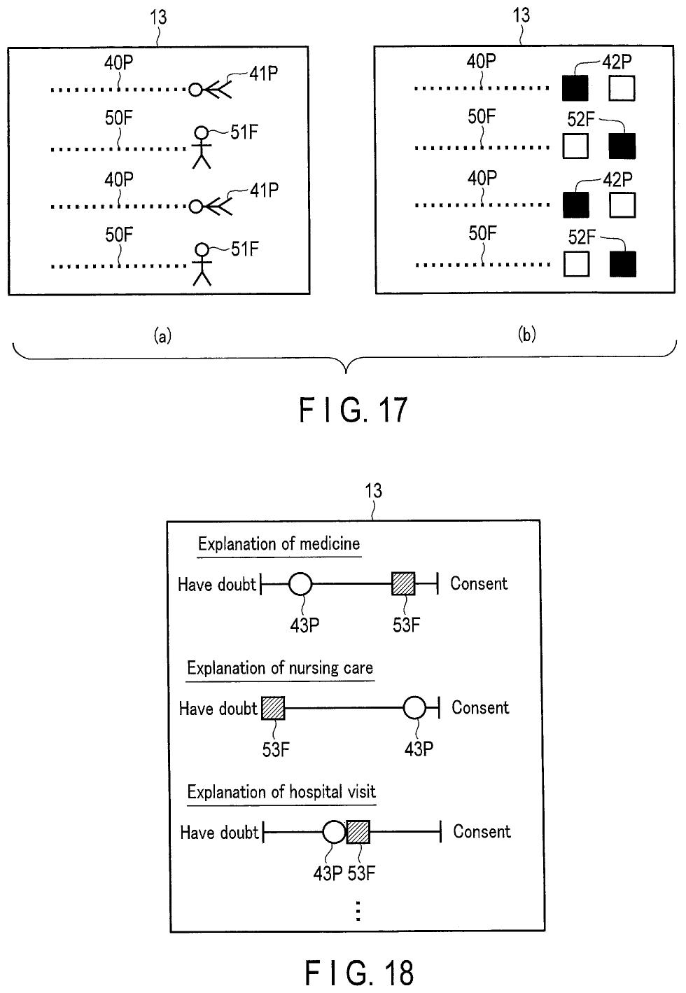

The patient entry field 40P together with the patient icon 41P and the family entry field 50F together with the family icon 51F may be alternately arranged closer to the left side of the screen along the vertical direction, as shown in FIG. 17(a), for example. The patient icon 41P and the family icon 51F may have different shapes and different colors. Also, among the entry fields arranged alternately along the vertical direction, the entry field for making an entry by pressing a patient button 42P may be the patient entry field 40P, and the entry field for making an entry by pressing a family button 52F may be the family entry field 50F, as shown in FIG. 17(b), for example.

The patient entry field and the family entry field are not limited to the instance where they occupy separate regions. For example, a point 43P indicating a patient's intent and a point 53F indicating a family's intent may be entered on a single axis, as shown in FIG. 18.

Although not illustrated, the patient entry field and the family entry field may be distinguished from each other with their positions varied from each other or may be distinguished from each other with their colors varied from each other. Alternatively, the patient entry field and the family entry field may be distinguished from each other with their fonts varied from each other or may be distinguished from each other with their sizes varied from each other. Alternatively, the patient entry field and the family entry field may be distinguished from each other with their designs varied from each other.

When making an entry into the patient entry field and the family entry field, the processing circuitry 15 may make either of them invisible. Namely, the processing circuitry 15 may display one of the patient entry field or the family entry field so that an entry can be made, and make the other of them invisible by not displaying it so that no entry can be made.

### Third Embodiment

Next, a family intent acquisition system which includes a family intent acquisition apparatus according to a third embodiment will be described.

The third embodiment is not limited to the instance of presenting the explanatory text D1x described above but includes an aspect of acquiring a patient's intent and a family's intent separately in a situation where an intent is input through voice.

Accordingly, the control function 15a of the processing circuitry 15 of the family intent acquisition apparatus 10 registers an external device for performing voice input from a patient. As the external device, any voice input device such as a microphone, a wearable terminal, etc., capable of performing wireless communication with the family intent acquisition apparatus 10 can be suitably used. The control function 15a is an example of a register unit.

The input interface 12 has a main unit microphone.

The intent acquisition function 15b of the processing circuitry 15 acquires an audio content input to the external device as a patient's intent, and acquires an audio content input to the main unit microphone as a family's intent.

The remaining aspects are the same as the first embodiment. The third embodiment may be applied to each modification of the first embodiment.

According to the above configuration, it is assumed that steps ST10 to ST30 are performed in the same manner as described above and that an intent entry screen for the explanatory text D1x is displayed on the display 13. Specifically, in step ST30, the processing circuitry 15 causes the display 13 to display a patient entry field for entering a patient's intent and a family entry field for entering a family's intent.

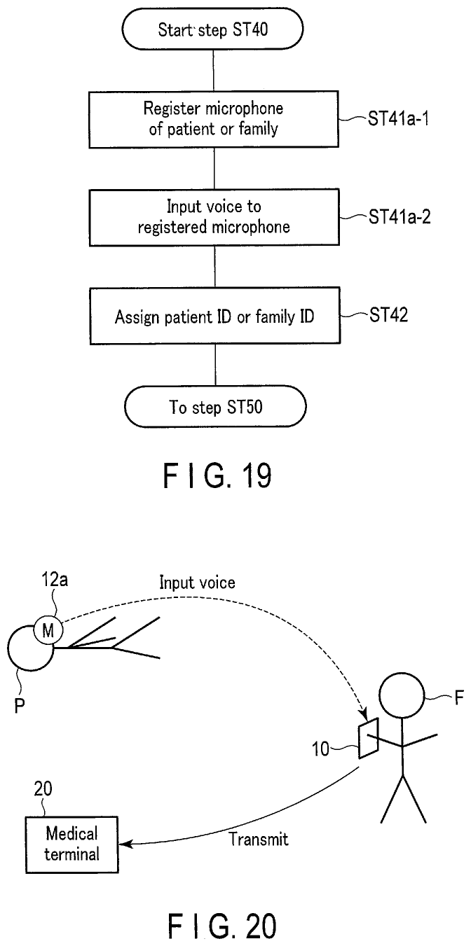

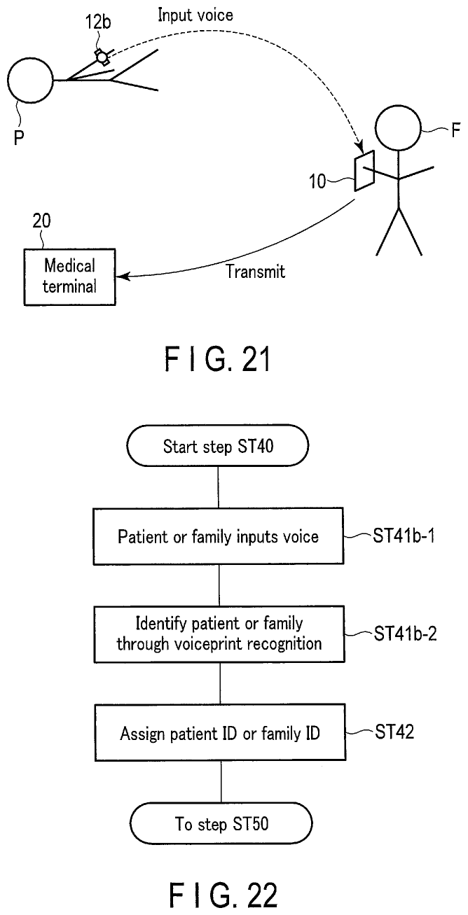

After step ST30, step ST40 consisting of steps ST41a-1, ST41a-2, and ST42 is performed, as shown in FIG. 19. It is assumed that the processing circuitry 15 registers a microphone of a patient or a family according to an operation performed by the patient or the family (step ST41a-1). As the microphone, a microphone 12a of a patient P is registered in association with the patient ID, as shown in FIG. 20, for example. A registered device is not limited thereto. A watch-type wearable terminal 12b of a patient P may be registered in association with the patient ID, as shown in FIG. 21, for example. However, the wearable terminal 12b is designed to enable voice input. Thereafter, the processing circuitry 15 acquires an audio content input to the registered microphone 12a as a patient's intent (step ST41a-2), and acquires an audio content input to the main unit microphone as a family's intent.

In step ST42 after step ST41a-2, the processing circuitry 15 assigns a patient ID to the acquired patient's intent and assigns a family ID to the acquired family's intent. Thereby, step ST40 is completed.

Thereafter, the processing of step ST50 and the processing of the subsequent steps are performed in the same manner as described above.

As described above, according to the third embodiment, an external device for performing voice input from a patient is registered. An audio content input to the external device is acquired as a patient's intent, and an audio content input to the main unit microphone is acquired as a family's intent. Therefore, in addition to the effects achieved by the first embodiment, it is possible to acquire a patient's intent and a family's intent separately based on the audio content input to the registered external device and the audio content input to the main unit microphone.

(Modification of Third Embodiment)

In the third embodiment, the microphone 12a and the watch-type wearable terminal 12b are shown as examples of the external device; however, the external device is not limited thereto. For example, the external device may be not only a watch-type wearable terminal but also a wearable terminal that is attached to an arm or a head, such as a glasses-type wearable terminal. The external device may also be a mobile terminal such as a smartphone so that a patient and a family can be distinguished from each other according to the input made to a chatting tool such as an SNS.

### Fourth Embodiment

Next, a family intent acquisition system which includes a family intent acquisition apparatus according to a fourth embodiment will be described.

The fourth embodiment is not limited to the instance of presenting the explanatory text D1x described above but includes an aspect of acquiring a patient's intent and a family's intent separately in a situation where an intent is input though voice.

Accordingly, the intent acquisition function 15b of the processing circuitry 15 of the family intent acquisition apparatus 10 performs voiceprint recognition on the audio content input to the main unit microphone of the input interface 12 to recognize patient's and family's voiceprints, thereby acquiring the input audio content corresponding to the patient's voiceprint as a patient's intent and acquiring the input audio content corresponding to the family's voiceprint as a family's intent.

The remaining aspects are the same as the first embodiment. The fourth embodiment may be applied to each modification of the first embodiment.

According to the above configuration, it is assumed that steps ST10 to ST30 are performed in the same manner as described above and that an intent entry screen for the explanatory text D1x is displayed on the display 13. Specifically, in step ST30, the processing circuitry 15 causes the display 13 to display a patient entry field for entering a patient's intent and a family entry field for entering a family's intent.

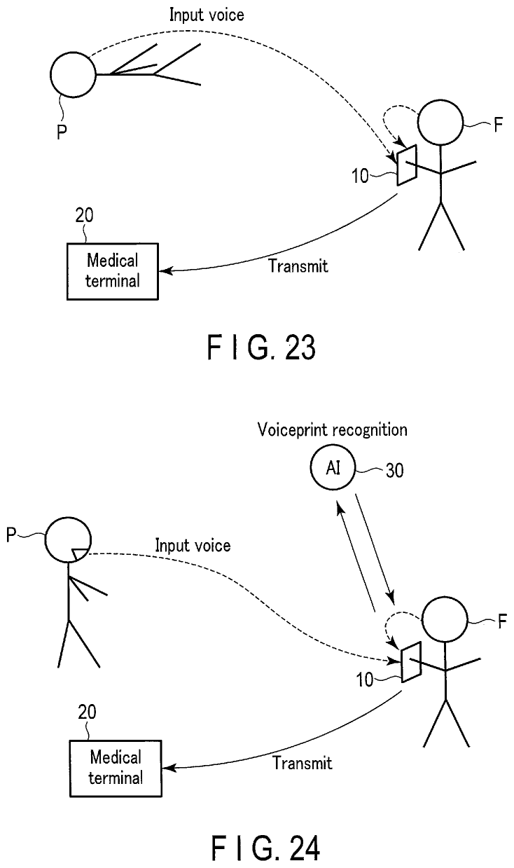

After step ST30, step ST40 consisting of steps ST41b-1, ST41b-2, and ST42 is performed, as shown in FIG. 22. According to an operation performed by a patient or a family, the processing circuitry 15 allows the patient or the family to perform voice input to the main unit microphone, as shown in FIG. 23 (step ST41b-1). The processing circuitry 15 performs voiceprint recognition on the audio content input to the main unit microphone to recognize patient's and family's voiceprints, and thereby identifies the patient or the family (step ST41b-2). Thereafter, the processing circuitry 15 acquires the input audio content corresponding to the patient's voiceprint as a patient's intent and acquires the input audio content corresponding to the family's voiceprint as a family's intent.

In step ST42 after step ST41b-2, the processing circuitry 15 assigns a patient ID to the acquired patient's intent and assigns a family ID to the acquired family's intent. Thereby, step ST40 is completed.

Thereafter, the processing of step ST50 and the processing of the subsequent steps are performed in the same manner as described above.

According to the fourth embodiment, voiceprint recognition is performed on the audio content input to the main unit microphone to recognize patient's and family's voiceprints, whereby the input audio content corresponding to the patient's voiceprint is acquired as a patient's intent and the input audio content corresponding to the family's voiceprint is acquired as a family's intent, as described above. Therefore, in addition to the effects achieved by the first embodiment, it is possible to acquire a patient's intent and a family's intent separately based on the audio content input from the patient whose voiceprint is recognized and the audio content input from the family whose voiceprint is recognized.

(Modification of Fourth Embodiment)

Although the family intent acquisition apparatus 10 performs voiceprint recognition in the fourth embodiment, the embodiment is not limited thereto. For example, the family intent acquisition apparatus 10 may transmit voice signals resulting from voice input into the main unit microphone to the external AI processing apparatus 30 and receive voiceprint recognition results from the AI processing apparatus 30, as shown in FIG. 24. In this case, it is possible to reduce the burden of performing voiceprint recognition on the family intent acquisition apparatus 10 in addition to achieving the effects achieved by the fourth embodiment.

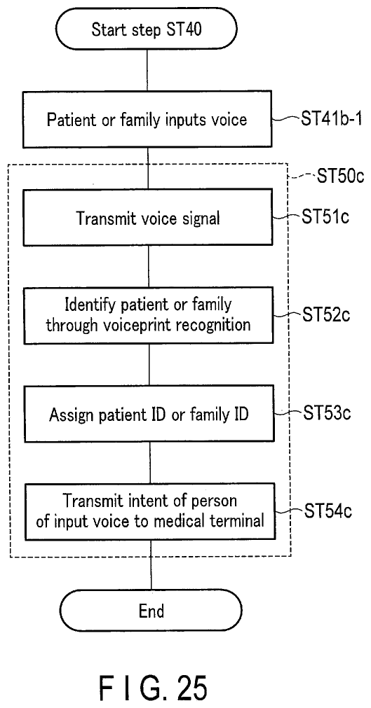

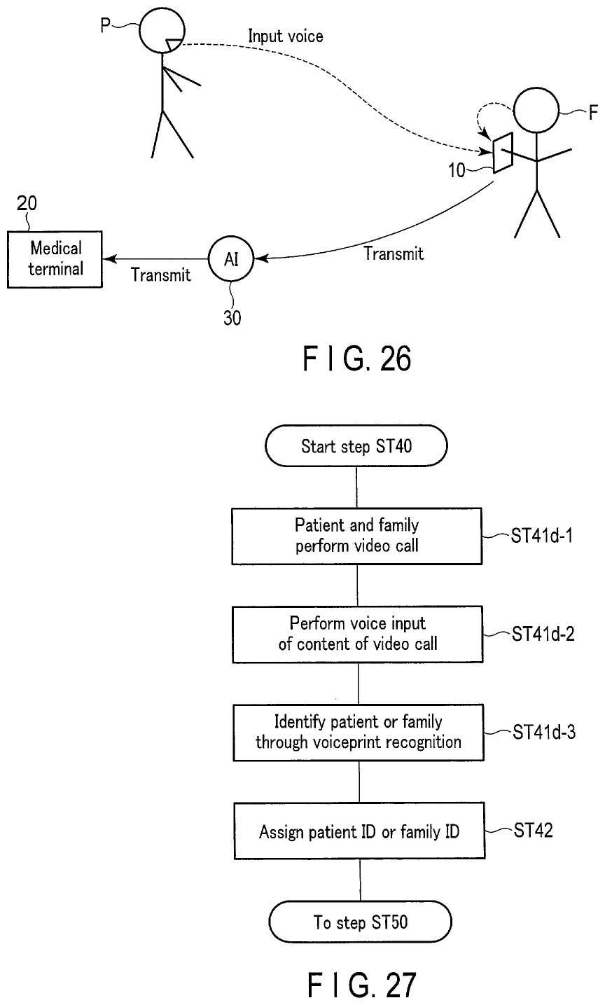

Alternatively, the family intent acquisition apparatus 10 may, for example, transmit voice signals resulting from voice input into the main unit microphone to the AI processing apparatus 30 on the communication network Nw, as shown in FIGS. 25 and 26 (step ST51c). In this case, the AI processing apparatus 30 identifies a patient or a family through voiceprint recognition (step ST52c), assigns a patient ID or a family ID according to the identification result (step ST53c), and thereby transmits an intent of a person performing voice input to the medical terminal 20 (step ST54c). In this case, it is possible to reduce the burden of the process ranging from voiceprint recognition to ID assignment on the family intent acquisition apparatus 10 in addition to achieving the effects achieved by the fourth embodiment.

Although voiceprint recognition is performed by a single family intent acquisition apparatus 10 in order to distinguish a patient's intent from a family's intent in the fourth embodiment, the embodiment is not limited thereto. For example, identification methods such as a speech pattern, a soundprint, fingerprint authentication of a tablet terminal, giving a name, an assistant function such as SiRi (Trademark), microphone directivity, fingerprint authentication of a pen-type input device or a mouse, and lip authentication by an indoor camera can be suitably adopted as a way to distinguish a patient's intent from a family's intent. As for a pen-type input device, it is possible to distinguish between a patient and a family not only through fingerprint authentication but also through detection of how it is held, where it is held, who holds it, etc., using a sensor or the like.

In addition, although the fourth embodiment does not particularly mention distinguishing a patient's intent from a family's intent, only a person who cares for or supports the patient most needs to be regarded as crucial, and there is no need to distinguish other family members. For example, if there are a patient A, a family member B who cares for the patient A most, and the other family members C, D, and E, the intent of the family member B is regarded as crucial, and the intents of the other family members C, D, and E may be collectively identified.

### Fifth Embodiment

Next, a family intent acquisition system which includes a family intent acquisition apparatus according to a fifth embodiment will be described.

The fifth embodiment is not limited to the instance of presenting the explanatory text D1x described above but includes an aspect of acquiring a patient's intent and a family's intent separately in a situation where an intent is input through voice.

Accordingly, while a patient with a patient terminal such as an ordinary smartphone and a family with the family intent acquisition apparatus 10 are making a video call, the intent acquisition function 15b of the processing circuitry 15 of the family intent acquisition apparatus 10 imports the audio content of the video call and recognizes the patient's and family's voiceprints. Thus, the intent acquisition function 15b acquires the audio content corresponding to the patient's voiceprint as a patient's intent and acquires the audio content corresponding to the family's voiceprint as a family's intent.

The remaining aspects are the same as the first embodiment. The fifth embodiment may be applied to each modification of the first embodiment.

According to the above configuration, it is assumed that steps ST10 to ST30 are performed in the same manner as described above and that an intent entry screen for the explanatory text D1x is displayed on the display 13. Specifically, in step ST30, the processing circuitry 15 causes the display 13 to display a patient entry field for entering a patient's intent and a family entry field for entering a family's intent.

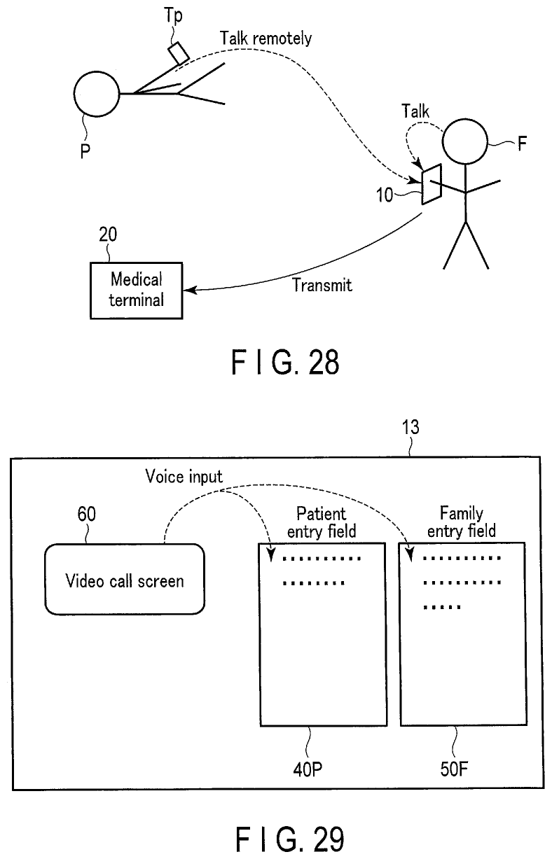

After step ST30, step ST40 consisting of steps ST41d-1 to ST41d-3 and ST42 are performed, as shown in FIG. 27. As shown in FIGS. 28 and 29, while a patient P with a patient terminal Tp and a family F with the family intent acquisition apparatus 10 are making a video call (step ST41d-1), the processing circuitry 15 performs voice input of the content of the video call by importing the audio content shown on a video call screen 60 (step ST41b-2). The processing circuitry 15 performs voiceprint recognition on the audio content of the video call to recognize patient's and family's voiceprints, and thereby identifies the patient or the family (step ST41d-3). Thereafter, the processing circuitry 15 inputs the audio content corresponding to the patient's voiceprint into the patient entry field 40P and also acquires it as a patient's intent. In addition, the processing circuitry 15 inputs the audio content corresponding to the family's voiceprint into the family entry field 50F and also acquires it as a family's intent.

In step ST42 after step ST41d-3, the processing circuitry 15 assigns a patient ID to the acquired patient's intent and assigns a family ID to the acquired family's intent. Thereby, step ST40 is completed.

Thereafter, the processing of step ST50 and the processing of the subsequent steps are performed in the same manner as described above.

According to the fifth embodiment, while a patient and a family are making a video call, the audio content of the video call is imported, and the patient's and family's voiceprints are recognized, whereby the audio content corresponding to the patient's voiceprint is acquired as a patient's intent and the audio content corresponding to the family's voiceprint is acquired as a family's intent, as described above. Therefore, in addition to the effects achieved by the first embodiment, it is possible to acquire a patient's intent and a family's intent separately through voiceprint recognition from the audio content in a daily video call.

(Modification of Fifth Embodiment)

In the fifth embodiment, an audio content of a video call is subjected to voiceprint recognition, whereby a patient's intent or a family's intent is acquired; however, the embodiment is not limited thereto. For example, an audio content related to medical care and an audio content unrelated to medical care may be sorted during a video call based on the presence or absence of medical terms, for example, so that only the audio content related to medical care is subjected to voiceprint recognition and acquired as a patient's intent or a family's intent. In this case, a patient and a family may talk about a topic unrelated to medical care during a video call, which makes it easy for them to use a video call. Also, the call content unrelated to medical care is not transmitted to the medical terminal 20, thus achieving protection of the patient's and family's privacy.

### Sixth Embodiment

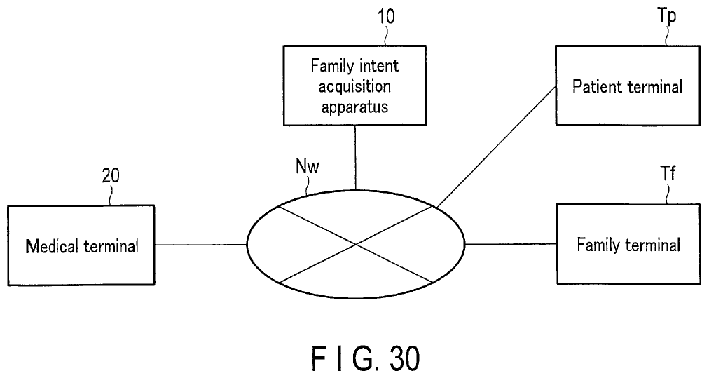

In a sixth embodiment, the family intent acquisition apparatus 10 is arranged on the communication network Nw between the medical terminal 20 and the patient terminal Tp as well as a family terminal Tf, as shown in FIG. 30. Specifically, the sixth embodiment includes performance of a process of producing an explanatory text D1x by performing the processing relating to a special note on an explanatory text D1 transmitted from the medical terminal 20, and a process of summarizing a patient's intent acquired from the patient terminal Tp and a family's intent acquired from the family terminal Tf. The process of producing an explanatory text D1x may be performed by the medical terminal 20. The patient terminal Tp and the family terminal Tf are ordinary communicable mobile devices such as a smartphone.

Accordingly, the processing circuitry 15 of the family intent acquisition apparatus 10 performs the processing relating to a special note on the explanatory text D1 to produce the explanatory text D1x. Note that the processing circuitry 15 transmits the produced explanatory text D1x to the patient terminal Tp and the family terminal Tf. In addition, the processing circuitry 15 summarizes the patient's intent acquired from the patient terminal Tp and the family's intent acquired from the family terminal Tf as intent information.

The remaining aspects are the same as the first embodiment.

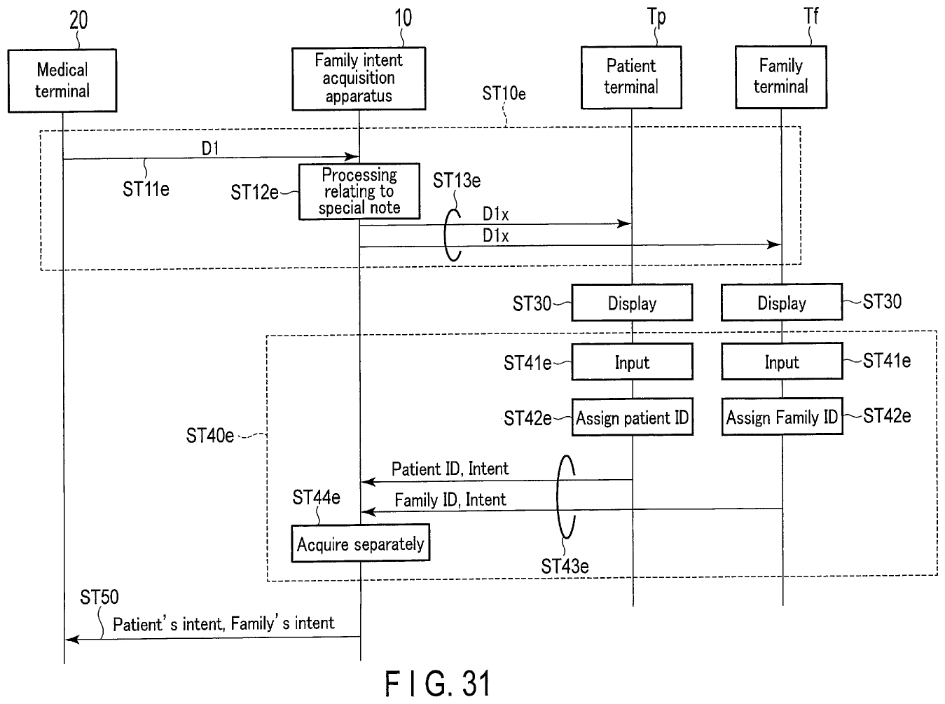

The sixth embodiment described above differs from the first embodiment in that an operation of step ST10e is performed in place of step ST10 and an operation of step ST40e is performed in place of step ST40, as shown in FIG. 31. Step ST10e consists of step ST11e to ST13e, and step ST40e consists of step ST41e to ST44e.

First, the processing circuitry 25 of the medical terminal 20, for example, produces the explanatory text D1 according to an operation performed by the medical staff and stores it in the memory 21. The processing circuitry 25 also transmits the explanatory text D1 stored in the memory 21 to the family intent acquisition apparatus 10 to be directed to the patient terminal Tp and the family terminal Tf (step ST11e).

The family intent acquisition apparatus 10 receives the explanatory text D1 transmitted to the patient terminal Tp and the family terminal Tf, and then generates an item of a special note with which a family is concerned from the explanatory text D1 based the explanatory text D1, followed by performance of the processing relating to a special note on the explanatory text D1 based on the generated item to produce the explanatory text D1x, in the same manner as described above (step ST12e). Thereafter, the family intent acquisition apparatus 10 transmits the produced explanatory text D1x to the patient terminal Tp and the family terminal Tf (step ST13e). Thereby, step ST10e consisting of steps ST11e to ST13e is completed.

In step ST30 after step ST10e, the patient terminal Tp and the family terminal Tf cause a display to display the received explanatory text D1x. The patient terminal Tp and the family terminal Tf also cause a display to display an intent entry screen for the explanatory text D1x.

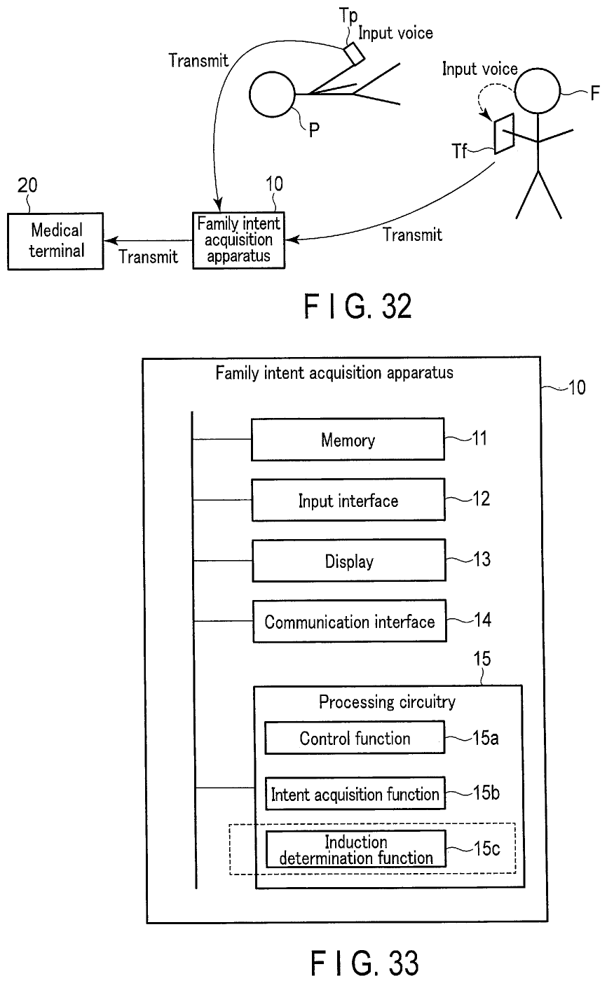

In step ST41e after step ST30, the patient terminal Tp inputs a patient's intent via voice according to an operation performed by the patient, as shown in FIG. 32. Also, the family terminal Tf inputs a family's intent via voice according to an operation performed by the family. The family terminal Tf updates the check box Ck of the explanatory text D1x according to an operation performed on the explanatory text D1x by the family, and acquires the family's intent according to an operation performed on the entry screen by the family.

In step ST42e after step ST41e, the patient terminal Tp assigns a patient ID to the patient's intent acquired through voice input. Also, the family terminal Tf assigns a family ID to the family's intent acquired through voice input.

In step ST43e after step ST42e, the patient terminal Tp transmits the intent assigned with a patient ID to the family intent acquisition apparatus 10 to be directed to the medical terminal 20. Also, the family terminal Tf transmits the explanatory text D1x with the check box Ck updated and the intent assigned with a family ID to the family intent acquisition apparatus 10 to be directed to the medical terminal 20. However, the explanatory text D1x with the check box Ck updated may be omitted, as also described above.

In step ST44e after step ST43e, the family intent acquisition apparatus 10 receives the patient's intent assigned with a patient ID from the patient terminal Tp. Also, the family intent acquisition apparatus 10 receives the family's intent assigned with a family ID and the explanatory text D1x with the check box Ck updated from the family terminal Tf. Thereby, the processing circuitry 15 of the family intent acquisition apparatus 10 acquires the patient's intent assigned with a patient ID and the family's intent assigned with a family ID separately. Also, the processing circuitry 15 produces intent information including the patient's intent assigned with a patient ID, the family's intent assigned with a family ID, and the explanatory text D1x with the check box Ck updated. Thereby, step ST40e consisting of steps 41e to 44e is completed.

In step ST50 after step ST40e, the processing circuitry 15 transmits the intent information to the medical terminal 20.

After step ST50, the medical terminal 20 causes the display 23 to display the patient's intent and the family's intent in such a manner as to distinguish them based on the information on the intent received. Thus, medical staff can understand the patient's intent and the family's intent separately.

According to the sixth embodiment described above, the family intent acquisition apparatus 10 on the communication network Nw produces an explanatory text D1x by performing the processing relating to a special note on an explanatory text D1 transmitted from the medical terminal 20, and summarizes a patient's intent acquired from the patient terminal Tp and a family's intent acquired from the family terminal Tf. Therefore, in addition to the effects achieved by the first embodiment, it is possible to realize a family intent acquisition apparatus without installing the family intent acquisition apparatus 10 in the patient terminal Tp and the family terminal Tf.

### Seventh Embodiment

Next, a family intent acquisition system which includes a family intent acquisition apparatus according to a seventh embodiment will be described.

In the seventh embodiment, it is possible to know whether a patient's intent is induced by a family's intent. For example, it is desirable that a patient's intent be decided by a patient himself/herself so that the patient's will is respected. If the patient's intent is an opinion or a will induced by a family and if the patient does not fully consent or have some doubt, it is desirable to make the feeling of strangeness noticeable. However, even if the patient's intent is an opinion or a will induced by a family, if the patient fully consents, no problem arises; thus, it is desirable that the patient be able to indicate by himself/herself that s/he consents.

Accordingly, the processing circuitry 15 of the family intent acquisition apparatus 10 includes an induction determination function 15c, as shown in FIG. 33.

The induction determination function 15c determines whether a patient's intent is induced by a family's intent or not by analyzing the patient's intent and the family's intent. This determination is performed by, for example, analyzing a conversation between a patient and a family. For example, a record is made of approximately how many minutes the family and the patient talked about each of the options. This makes it possible to know whether all the options have been explained and discussed or not and whether the allocation of time for all the options is appropriate or not. For example, if only a certain option is talked about by a family, and the other options are not, it is highly likely that the patient's intent is induced by the family's intent. Also, if a family repeatedly asks “Do you have a stomachache?”, for example, the patient is likely to have a stomachache due to suggestion. Likewise, if a family keeps saying “You are fine.”, the patient is likely to feel fine due to being numb to the pain due to suggestion. In this manner, the induction determination function 15c records the amount of time that a family spends speaking and an imbalance in the speech content by analyzing the conversation, and determines whether the induction occurs or not based on the imbalance in the family's speech.

If the result of the determination shows that the patient's intent is induced, the induction determination function 15c may determine whether the induced intent indicates consent or not. In this case, the induction determination function 15c infers the degree of consent of the induced intent, and if the degree of consent exceeds a threshold, the induction determination function 15c may determine that the induced intent indicates consent. The induction determination function 15c is an example of a first determination unit and a second determination unit.

The control function 15a of the processing circuitry 15 transmits intent information further including the results of each determination.

The remaining aspects are the same as the fourth embodiment. The seventh embodiment may be applied to the third, fifth, and sixth embodiments relating to voice input. The seventh embodiment may also be applied to the first or second embodiment.

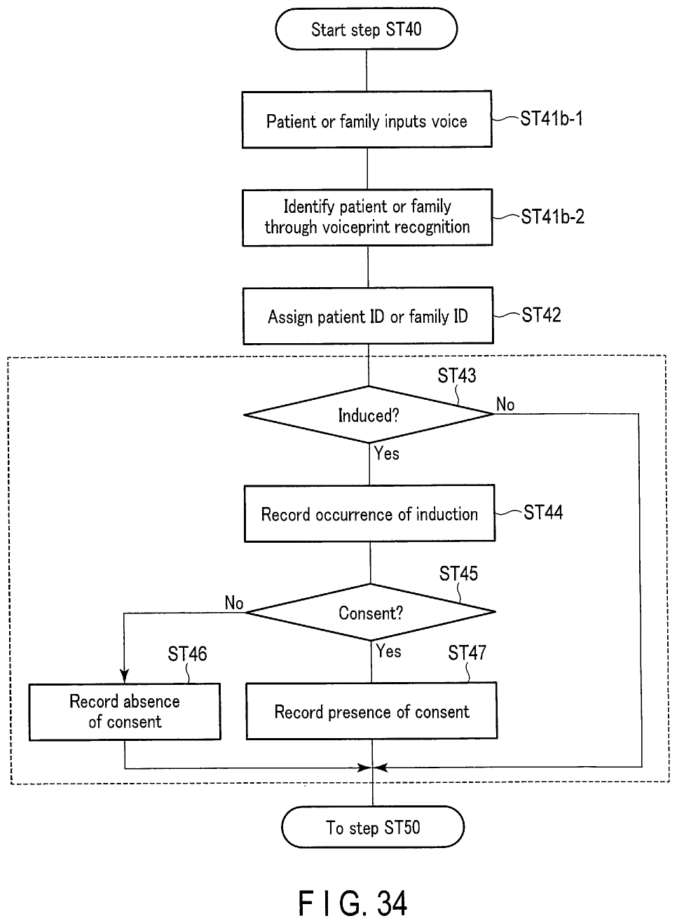

According to the configuration described above, steps ST10 to ST30, ST41b-1, ST41b-2, and ST42 are performed in the same manner as described above, as shown in FIG. 34, and an input audio content corresponding to the patient's voiceprint is acquired as a patient's intent and assigned with a patient ID. Also, an input audio content corresponding to the family's voiceprint is acquired as a family's intent and assigned with a family ID. Thereby, step ST42 is completed.

After step ST42, the processing circuitry 15 of the family intent acquisition apparatus 10 determines whether a patient's intent is induced by a family's intent or not by analyzing the patient's intent and the family's intent (step ST43). If the determination result indicates no occurrence of induction, step ST40 ends and the process moves to step ST50.

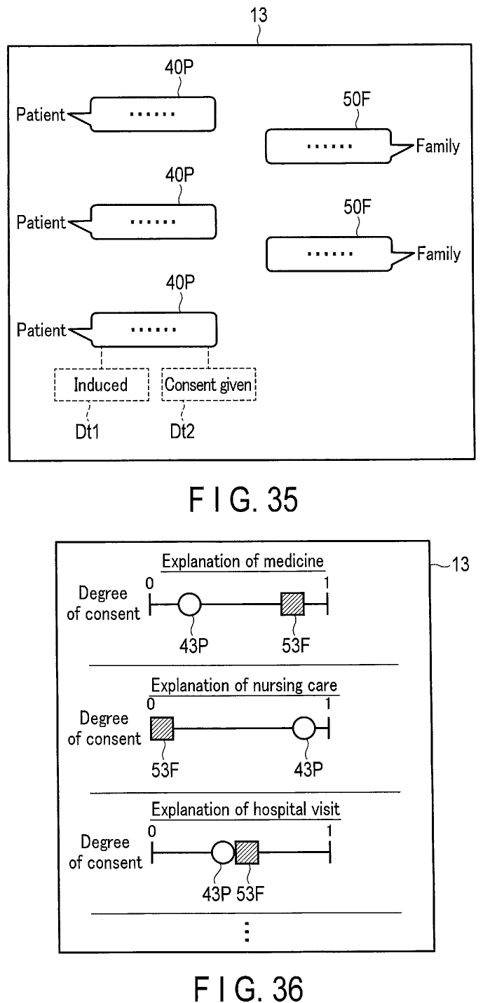

If the determination result of step ST43 indicates that the patient's intent is induced, the processing circuitry 15 records the occurrence of induction in the memory 11 (step ST44). Also, the processing circuitry 15 causes the display 13 to display a determination result Dt1 indicating “Induced” in association with the induced intent, as shown in FIG. 35.

Subsequently, the processing circuitry 15 determines whether the induced intent indicates consent or not (step ST45). For example, the processing circuitry 15 infers the degree of consent of the induced intent, and determines that the induced intent indicates consent if the degree of consent exceeds a threshold. If the determination result does not indicate consent, the processing circuitry 15 records the absence of consent in the memory 11 (step ST46). Although not illustrated, the processing circuitry 15 causes the display 13 to display the determination result indicating “Absence of consent” in association with the intent for which no consent is obtained, and ends step ST40 to move on to step ST50. If the determination result of step ST45 indicates consent, the processing circuitry 15 records the presence of consent in the memory 11 (step ST47). Also, the processing circuitry 15 causes the display 13 to display a determination result Dt2 indicating “Presence of consent” in association with the intent for which consent is obtained, and ends step ST40 to move to step ST50, as shown in FIG. 35.

In step ST50 after step ST40, the processing circuitry 15 transmits, to the medical terminal 20, intent information including the intent assigned with a patient ID, the intent assigned with a family ID, and the determination result of whether the patient's intent is induced by the family's intent or not. If the patient's intent is induced, the intent information is transmitted while further including the determination result of whether the induced intent indicates consent or not.

According to the seventh embodiment, it is determined whether a patient's intent is induced by a family's intent or not by analyzing the patient's intent and the family's intent, and intent information further including the determination result is transmitted, as described above. Therefore, in addition to the effects achieved by the fourth embodiment, it is possible to notify medical staff whether the patient's intent is induced or not.

Also, according to the seventh embodiment, if the determination result shows that the patient's intent is induced, a determination is made as to whether the induced intent indicates consent or not, and intent information further including the determination result is transmitted. Therefore, in addition to the above-described effects, it is possible to notify medical staff whether the patient consents to the induced patient's intent or not.

Further, according to the seventh embodiment, the degree of consent of the induced intent is inferred, and if the degree of consent exceeds a threshold, it is determined that the induced intent indicates consent. Therefore, in addition to the above-described effects, it is possible to make a determination regarding consent based on clear criteria for determination, which are a quantified degree of consent and a threshold.

(Modification of Seventh Embodiment)

In the seventh embodiment, a quantified degree of consent is not displayed; however, the embodiment is not limited thereto. For example, a point 43P indicating a patient's degree of consent and a point 53F indicating a family's degree of consent may be displayed on a single axis on the display 13, as shown in FIG. 36. The point 43P indicating a patient's degree of consent may be input according to an operation performed by a patient, or may be a point indicating a degree of consent of an induced intent determined by the induction determination function 15c. Also, the point 53F indicating a family's degree of consent is input according to an operation performed by a family. In these cases, it is possible to quantitatively display patient's and family's degree of consent, in addition to the effects achieved by the seventh embodiment; thus, it can be expected to prompt a discussion between a patient and a family.

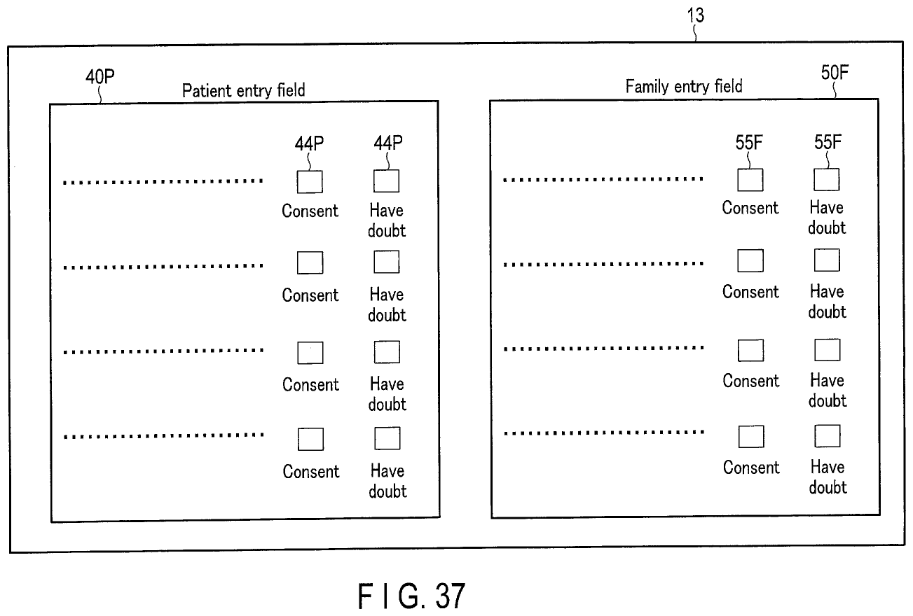

In the seventh embodiment, the result of a determination regarding consent made by the induction determination function 15c is displayed; however, the embodiment is not limited thereto. For example, for a patient's intent in the patient entry field 40P and a family's intent in the family entry field 50F, respectively, GUIs 44P and 44F which receive input indicating the presence or absence of consent to the intent may be provided, as shown in FIG. 37. In FIG. 37, the GUIs 44P and 55F associated with “Consent” receive input indicating the presence of consent. Also, the GUIs 44P and 55F associated with “Have doubt” receive input indicating the absence of consent. The GUIs 44P and 55F are an example of a reception unit. In this case, the processing circuitry 15 transmits, to the medical terminal 20, intent information further including the presence or absence of consent for which input is received. In this case, it is possible to input the presence or absence of patient's and family's consent, in addition to the effects achieved by the seventh embodiment; thus, it can be expected to prevent inconsistency between the result of the determination on whether consent is obtained or not and whether consent is actually obtained or not.

In the seventh embodiment, the patient's intent and the family's intent are not weighted; however, the embodiment is not limited thereto. For example, if a patient who rarely reports pain does so, it is recorded with increased weighting even if a family indicates “fine”. Also, if a patient says “fine” and a family makes a big deal, the patient's intent is recorded with the weighting of the patient's intent reduced, and the family's intent is recorded with the weighting of the family's intent increased. In these cases, it is possible to achieve objectivity and quantification of the intent and efficiently confirm a family's will, in addition to the effects described above.

According to at least one embodiment described above, an intent of a patient and an intent of a family who cares for or supports the patient are acquired. In addition, the patient's intent is assigned with a patient ID, and the family's intent is assigned with a family ID. Furthermore, intent information including the intent assigned with a patient ID and the intent assigned with a family ID is transmitted. Therefore, it is possible to acquire the patient's intent and the family's intent separately.

The terminology “processor” used herein refers to, for example, a central processing unit (CPU), a graphics processing unit (GPU), or circuitry such as an application-specific integrated circuit (ASIC), a programmable logic device (such as a simple programmable logic device (SPLD), a complex programmable logic device (CPLD), or a field programmable gate array (FPGA), and so on. If the processor is a CPU, for example, the processor implements its function by reading and executing the program stored in storage circuitry. On the other hand, if the processor is an ASIC, for example, that function is directly incorporated in the circuitry of the processor as a logic circuit, instead of a program being stored in the storage circuitry. The processors described in connection with the above embodiments are not limited to single-circuit processors; a plurality of independent circuits may be integrated into a single processor that implements the functions. Furthermore, the plurality of components illustrated in FIG. 1, 8, or 33 may be integrated into a single processor to implement the functions.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A family intent acquisition apparatus, comprising processing circuitry, the processing circuitry being configured to:
acquire an intent of a patient and an intent of a family who cares for or supports the patient;
assign the intent of the patient with a patient ID, and assign the intent of the family with a family ID; and
transmit intent information including the intent assigned with the patient ID and the intent assigned with the family ID.

2. The family intent acquisition apparatus according to claim 1, wherein the processing circuitry is further configured to:
cause a display to display a patient entry field for entering the intent of the patient and a family entry field for entering the intent of the family;
acquire a content entered into the patient entry field as the intent of the patient; and
acquire a content entered into the family entry field as the intent of the family.

3. The family intent acquisition apparatus according to claim 1, further comprising a main unit microphone,
wherein the processing circuitry is further configured to:
register an external device for performing voice input from the patient;
acquire an audio content input to the external device as the intent of the patient; and
acquire an audio content input to the main unit microphone as the intent of the family.

4. The family intent acquisition apparatus according to claim 1, comprising a main unit microphone,
wherein the processing circuitry is configured to:
perform voiceprint recognition on an audio content input to the main unit microphone to recognize voiceprints of the patient and the family, and thereby acquire an input audio content corresponding to the voiceprint of the patient as the intent of the patient and acquire an input audio content corresponding to the voiceprint of the family as the intent of the family.

5. The family intent acquisition apparatus according to claim 1, wherein the processing circuitry is configured to:
while the patient and the family are making a video call, import an audio content of the video call and perform voiceprint recognition of the patient and the family, and thereby acquire an audio content corresponding to a voiceprint of the patient as the intent of the patient and acquire an audio content corresponding to a voiceprint of the family as the intent of the family.

6. The family intent acquisition apparatus according to claim 3, wherein the processing circuitry is further configured to:
perform first determination processing of determining whether the intent of the patient is induced by the intent of the family by analyzing the intent of the patient and the intent of the family; and
transmit the intent information further including a result of the determination.

7. The family intent acquisition apparatus according to claim 6, wherein the processing circuitry is configured to:
if a result of the determination shows that the intent of the patient is induced, perform second determination processing of determining whether the induced intent indicates consent; and
transmit the intent information further including a result of the determination performed by the second determination processing.

8. The family intent acquisition apparatus according to claim 7, wherein the processing circuitry is configured to, in the second determination processing, infer a degree of consent of the induced intent, and if the degree of consent exceeds a threshold, determine that the induced intent indicates consent.

9. The family intent acquisition apparatus according to claim 8, wherein the processing circuitry is further configured to cause a display to display the degree of consent of the induced intent.

10. The family intent acquisition apparatus according to claim 6, wherein the processing circuitry is further configured to,
if the processing circuitry receives input indicating a presence or absence of consent to the intent for each of the intent of the patient and the intent of the family,
transmit the intent information further including the received input indicating the presence or absence of consent.

11. The family intent acquisition apparatus according to claim 4, wherein the processing circuitry is further configured to:
perform first determination processing of determining whether the intent of the patient is induced by the intent of the family by analyzing the intent of the patient and the intent of the family; and
transmit the intent information further including a result of the determination.

12. The family intent acquisition apparatus according to claim 11, wherein the processing circuitry is configured to:
if a result of the determination shows that the intent of the patient is induced, perform second determination processing of determining whether the induced intent indicates consent; and
transmit the intent information further including a result of the determination performed by the second determination processing.

13. The family intent acquisition apparatus according to claim 12, wherein the processing circuitry is configured to, in the second determination processing, infer a degree of consent of the induced intent, and if the degree of consent exceeds a threshold, determine that the induced intent indicates consent.

14. The family intent acquisition apparatus according to claim 13, wherein the processing circuitry is further configured to cause a display to display the degree of consent of the induced intent.

15. The family intent acquisition apparatus according to claim 11, wherein the processing circuitry is further configured to,
if the processing circuitry receives input indicating a presence or absence of consent to the intent for each of the intent of the patient and the intent of the family,
transmit the intent information further including the received input indicating the presence or absence of consent.

16. The family intent acquisition apparatus according to claim 5, wherein the processing circuitry is further configured to:
perform first determination processing of determining whether the intent of the patient is induced by the intent of the family by analyzing the intent of the patient and the intent of the family; and
transmit the intent information further including a result of the determination.

17. The family intent acquisition apparatus according to claim 16, wherein the processing circuitry is configured to:
if a result of the determination shows that the intent of the patient is induced, perform second determination processing of determining whether the induced intent indicates consent; and
transmit the intent information further including a result of the determination performed by the second determination processing.

18. The family intent acquisition apparatus according to claim 17, wherein the processing circuitry is configured to, in the second determination processing, infer a degree of consent of the induced intent, and if the degree of consent exceeds a threshold, determine that the induced intent indicates consent.

19. The family intent acquisition apparatus according to claim 18, wherein the processing circuitry is further configured to cause a display to display the degree of consent of the induced intent.

20. The family intent acquisition apparatus according to claim 16, wherein the processing circuitry is further configured to,
if the processing circuitry receives input indicating a presence or absence of consent to the intent for each of the intent of the patient and the intent of the family,
transmit the intent information further including the received input indicating the presence or absence of consent.
